(19) United States
(12) Patent Application Publication
(10) Pub. No.: US 2010/0004678 A1
(43) Pub. Date: Jan. 7, 2010

(54) AMBIDEXTROUS GRASPING SYSTEM FOR A MEDICAL INSTRUMENT

(76) Inventor: Valeria Querol Garica, Mexico City (MX)

Correspondence Address: DOWELL & DOWELL P.C., 103 Oronoco St., Suite 220, Alexandria, VA, 22314, US

(21) Appl. No.: 12/310,225

(22) PCT Filed: Aug. 18, 2006
(86) PCT No.: PCT/IB2006/002245

§ 371 Date: Feb. 18, 2009

(51) Int. Cl. A61B 17/30

(52) U.S. Cl. 606/210

(57) ABSTRACT

A medical instrument with an ambidextrous grasping system, which includes: a single elongate arm and a double elongate arm, which are joined to one another by a joining means at the rear ends, a sliding blocking mechanism which is connected and disconnected by means of a vertical movement, and tips for medical use which are located at the front end, wherein the blocking mechanism is activated by slight pressure exerted by the user in the grasping area, in order to activate and deactivate the mechanism, whilst maintaining a constant distance between the two arms in the grasping area, in order to improve the comfort of the user; the said instrument with a grasping system does not have a pivoting point, thus facilitating its cleaning, maintenance and use, and in addition this grasping system is simple to manufacture and has a low cost.

### TECHNICAL FIELD OF THE INVENTION

The application field of the present invention is related to the industry dedicated to the manufacturing and the commercial trading of instruments for medical purposes.

### BACKGROUND OF INVENTION

The present invention is related to a new grasping system for a medical instrument that allows both, right handers and left handers the use of it, with ease. Not only does this invention allow a comfort improvement and a better grasping and system for the user, but also gives the user an ergonomic benefit. This invention also provides a medical instrument that makes cleaning and maintenance easier.

There is a huge variety of instruments in the State of the Art describing medical instruments of this kind. Some of these are mentioned below.

In the U.S. Pat. No. 2,652,832 a needle holder formed by two arms with pointed frontal ends or tips designed to holding the needles, is mentioned. Such arms are united on two points, behind the tips by an axle or dual rear point and on the back side by a clinching. The needle holder includes a middle adapted area or point on the external faces of the arms for the user's grasping, and a blocking system on the internal faces of the arms.

The blocking mechanism allows the needle holder to maintain the tips face to face to hold the needle. When the blocking system is not disabled, the endings open to release the needle. This needle holder presents grasping problems, because when the blocking mechanism is activated, the distance between the internal faces of both arms is diminished, making the holding of the needles difficult. This invention presents another problem; this is that after some use it accumulates waste or residues of surgical material in the union, which makes the cleaning of it difficult. Besides this, after a period of time the material deposits in the axle or dual rear point generates a slow waste having to make maintenance actions in order to re-graduate the needle holder.

The U.S. Pat. No. 4,823,792 protects a grasping device and blocking mechanism for medical instruments, such as: pliers, needle holders and similar for surgery. Such a device is of similar configuration to the U.S. Pat. No. 2,652,832 and also includes two arms connected by an axle or dual rear point and means of a union on the rear end. The patent protects one system that joins and separates the device's arms on the axle or dual rear point, when the arms separate up to a maximum position, in order to make the cleaning process easy. All the above presents a problem, if the device reaches the maximum position during its use, it would fit out provoking the disassembling of the dice during its operation. Another problem that this invention presents is the complex manufacturing of the axle or dual rear parts, such as the assembling rectangular box and the swell that joins in this box. So, this invention presents disadvantages due to the fact that the swells are difficult parts to produce and raise the production costs. This patent also protects a blocking mechanism located in the middle point of the instrument, behind the axle or dual rear point, but before the means of joining the rear end. This blocking mechanism has the same operational system of the U.S. Pat. No. 2,652,832 and it presents the same grasping problems because when the blocking mechanism is activated, the distance between the internal faces of the arms is diminished, making the grasping of the instrument difficult.

The U.S. Pat. No. 4,800,880 protects an adapted surgical instrument to hold a needle that basically consists of a similar configuration to the one explained above of U.S. Pat. No. 2,652,832 and also includes two arms. These arms are also united by means of an axle or dual rear and by a spring connection next to the rear end of the instrument. In addition, it has a bolt mechanism in the middle point to fasten both arms in an open or closed position. This invention presents the problems explained before of grasping, due to the distance shortening between internal faces of the arms, when the bolt mechanism is activated. It also presents the maintenance problems due to the accumulation of surgical material in the axle or dual rear point.

The U.S. Pat. No. 5,104,397 protects a closing or blocking mechanism of a surgical instrument with almost the same configuration explained on U.S. Pat. No. 2,652,832 which possesses two connected arms by means of an axle or dual rear point. In addition, it has a closing mechanism located on the posterior part of the instrument that consists of different parts which are difficult to manufacture, such as swells, springs, etc.

As seen above the US patents described before share two main disadvantages: the existing point of an axle or dual rear point to join the arms that makes the cleaning and maintenance of the instrument a difficult task, and the blocking or closing mechanism is located behind the axle point provoking that the distance between arms is diminished on the closing or grasping position, making manipulation and the use of the instrument during its operation, uncomfortable.

Therefore, on the State of the Art there is the existing need of a medical grasping ambidextrous instrument that doesn't have an axle or dual rear point and can give comfort to the person who is using it, making cleaning, use, and maintenance, an easy task. Besides, it is desirable that such a grasping system is of low cost and simple to manufacture.

### BRIEF DESCRIPTION OF THE FIGURES

FIG. 1 corresponds to a right frontal top perspective view of the medical instrument that has a grasping system of the present invention, in a preferred embodiment in an open position.

FIG. 2 corresponds to a right side view, the medical instrument that has the grasping system on the present invention, in a preferred embodiment in an open position.

FIG. 3 corresponds to a right side view, the medical instrument that has the grasping system of the present invention, in a preferred embodiment in a closed position.

FIG. 4 corresponds to a top view of the medical instrument that has the grasping system of the present invention.

FIG. 5 corresponds to a bottom view of the medical instrument that has the grasping system of the present invention.

FIG. 6 corresponds to a right posterior perspective view in detail of a medical instrument that has the grasping system of the present invention, in a preferred embodiment in an open position.

FIG. 7 corresponds to a left rear top perspective view of a medical instrument that has the grasping system of the present invention, in a preferred embodiment in an open position.

FIG. 8 corresponds to a right side view of a medical instrument that has the grasping system of the present invention, in a preferred embodiment in an open position.

FIG. 9 corresponds to a right side view of a medical instrument that has the grasping system of the present invention, in a preferred embodiment in a closed position.

In order to make the comprehension of the present invention easier, table 1 presents reference numbers, components and characteristics mentioned on figures.

TABLE 1
REFERENCE NUMBERS

| NO. | COMPONENT | No. | COMPONENT |
|---|---|---|---|
| 10 | Medical instrument with | 11 | Frontal end of the |
| | grasping system | | instrument |
| 12 | First crossing point | 13 | Second crossing point |
| 14 | Rear end of the | 15 | Medical use tip |
| | instrument | | |
| 20 | Double elongated arm | 16 | Medical use tip |
| 21 | Front portion of the | 30 | Single elongated arm |
| | double arm | | |
| 31 | Front portion of the | 50 | Joining means of both arms |
| | single arm | | |
| 22 | Intermediate portion of | 32 | Intermediate portion of |
| | the double arm | | single arm |
| 24 | First curved portion of | 34 | First curved portion of |
| | the double arm | | single arm |
| 25 | Second curved portion of | 35 | Second curved portion of |
| 25′ | the double arm | | single arm |
| 26 | Double arm knurled | 36 | Single arm knurled grasping |
| 26′ | grasping | | |
| 27 | Interior surface of | 37 | Interior surface of single |
| | double arm in portion 22 | | arm in the portion 32 |
| 42 | First member of the | 41 | Second member of the |
| | blocking mechanism | | blocking system |
| 44 | Receiver end of the | 43 | Sliding protuberance of the |
| | blocking mechanism | | blocking mechanism |

### DETAILED DESCRIPTION OF THE PREFERRED EMBODIMENT

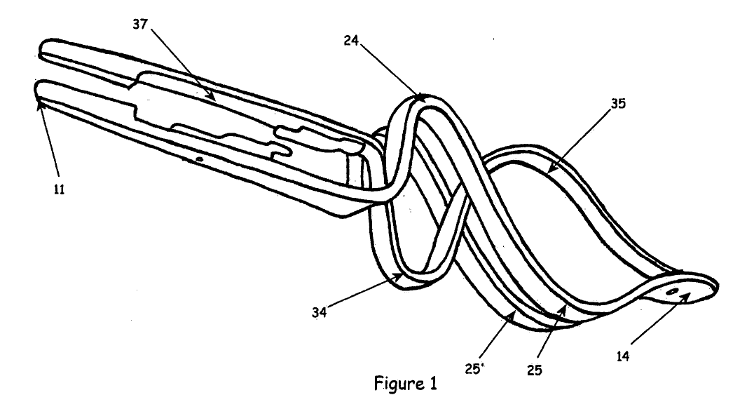

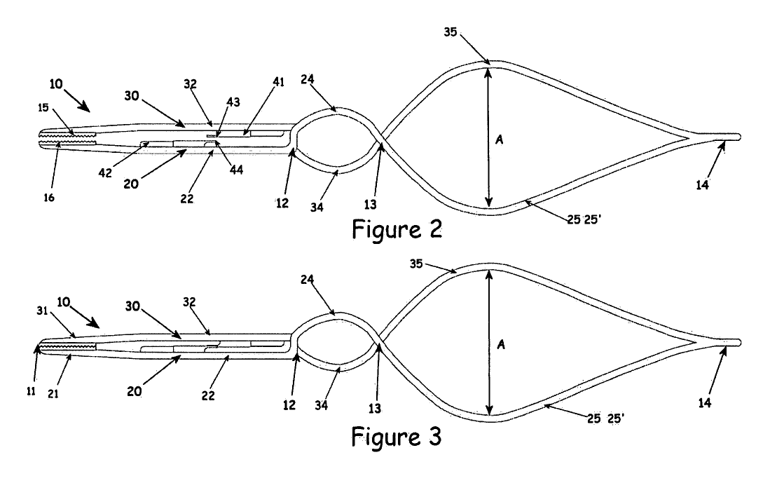

FIG. 1 shows the preferred embodiment of the present invention, which consists on a medical instrument 10 with a grasping system of the present invention formed by two arms: an elongated double arm 20 and an elongated single arm 30. As can be observed in FIGS. 2 and 3, the elongated double arm 20 is flat on frontal portion 21 and intermediate portion 22. From a first point, the arm 20 curves upward defining a first curved portion 24 on a “U” upside down form. From a second point, the arm 20 curves downward defining a second curved portion 25 on a “U” form, being the second curved portion more extended than the first curved portion. Finally, the arm 20 ends with a flat ending.

Likewise, the elongated single arm 30 is flat on frontal portions 31 and on intermediate portion 32. From a first point, the arm 30 curves downward defining a first curved portion 34 on a “U” form. From a second point, the arm 30 curves upward defining a second curved portion 35 on a “U” upside down form, being the second curved portion more extended than the first curved portion. Finally, the arm 30 ends with a flat ending. Second curved portions 25 and 35 of both arms, form the grasping area of the instrument.

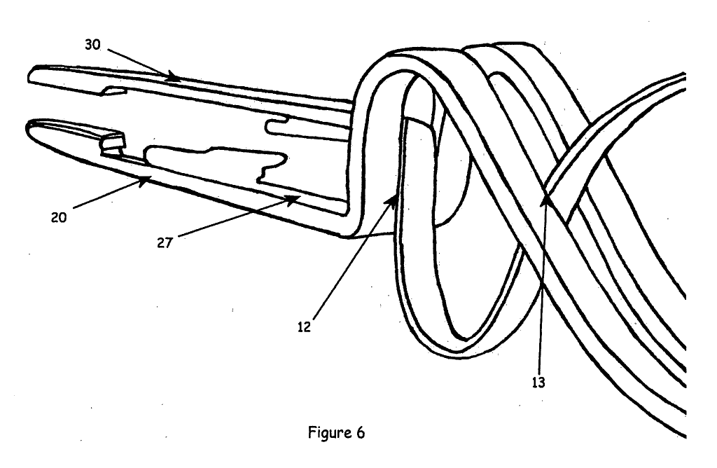

Referring to FIGS. 1 to 6, the double arm 20 has a frontal portion 21 and an intermediate portion 22 with only one branch line. The double arm 20 separates in two branch lines at the beginning of the first curved portion 24 on a “U” upside down forming a space between both branch lines. The single elongated arm 30 passes through the space between both branch lines generating a first crossing point 12. The single elongated arm 30 passes again through the space formed between both branch lines of the double arm 20 generating a second crossing point 13. The presence of both branch lines of the double arm in the grasping area 25, 25′ allows a better holding and grasping to the user, as well as, a better control of the instrument. Specifically, it is noticed in FIGS. 1 to 6, that none of the arms interferes, nor scratches, nor rubs as passing through the crossing points 12 and 13.

The medical instrument also has a frontal end 11 where the front portions 21 and 31 hold tips 15 and 16 for medical use. The medical instrument also has a rear end 14 where both internal surfaces of the arms 20 and 30 meet; and joining means 50 are used for fastening them in the desired position, as seen in FIG. 5. Such joining means 50 can be bolts, clinching gears, or fastening screws, being able to use other joining means. In addition, the preferred embodiment of the present invention comprises a flux material that covers the entire rear end 14 and fastens both arms. Such flux material can be removed easily during maintenance and when graduating the instrument.

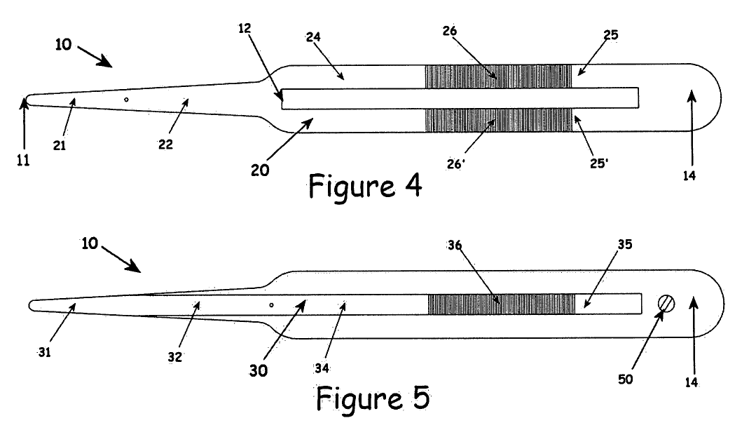

FIG. 4 shows the preferred embodiment that the present invention presents, on the external surface of the grasping area 25, 25′ of the double arm 20 a knurled grasping 26, 26′ to make the grasping and holding of the instrument easier. In a similar way, FIG. 5 shows that in the grasping area 35 the external surface of the single arm 30 presents a knurled grasping 36 to make the instrument grasping and holding easier.

The preferred embodiment of the present invention also has on the intermediate portions 22 and 32 of both arms, a blocking mechanism as the ones known in the State of the Art. The purpose of this mechanism is to maintain both ends 15 and 16 together to maintain the desired holding pressure on any object to be held. As seen in FIGS. 1 and 2, this blocking mechanism is made of a first member 42 that extents at right angles or perpendicularly to the internal surface 27 of the intermediate portion 22 and curves to remain parallel to the double arm 20. The first member 42 ends with a receiver end 44. The blocking mechanism also includes a second member 41 that comes out perpendicularly towards the internal surface 37 of the intermediate portion 32 and curves to remain parallel to the single arm 30. The second member 41 ends with a sliding protuberance 43 that fits in the receiver end 44.

The preferred embodiment of the present invention has two basic positions: the open position where the tips 15 and 16 are separated as shown in FIGS. 1 and 2; and closed position where the tips 15 and 16 are joined, as shown in FIG. 3, in order to hold any object.

For the operation of the preferred embodiment of the present invention, the user should make a light pressure on the knurled grasping areas 26, 26′ and 36 of both arms. This pressure is transformed in a vertical movement of one arm in relation to the other, performing its point of support in the rear end 14. Such movement is transmitted from the grasping area 25, 25′ and 35 where the pressure is performed towards the intermediate portions 22 and 32 where the blocking mechanism is installed. The vertical force of this movement brings the members 41 and 42 closer one to another until the receiver end 44 and the sliding protuberance 43 fit together to stay in the closed position. Likewise a slight vertical pressure on the grasping area 25, 25′ and 35 unfits the elements of the blocking mechanism, thus separating the tips 15 and 16 and releasing the object that could be found between them, to remain in open position. It is important to emphasize that during movement, in none of the crossing points 12 or 13 the material of the arms makes contact or rub. Another point to emphasize is that during movement, the distance (A) on the grasping area suffers a minimal change, as shown in FIGS. 2 and 3. This enables the user to keep a constant grasp on the instrument, thus giving an ergonomic advantage during its use.

The present invention can be easily used by right or left handed people. Due to the fact that in order to activate or deactivate the blocking mechanism only a light pressure must be done, without having to do side movements that are more difficult for left handers than to right handers.

Also, the exclusion of a gear or pivot like the one used in the present surgical instruments, makes cleaning and maintenance easier.

One more advantage of the described invention is the possibility of introducing and manipulating the frontal portion 21 and 31 and the intermediate portion 22 and 32 of the instrument through the surgical field during a surgery. The above due to the change of distance between both arms 20 and 30 or such portions almost constantly when the instrument is activated from open position to closed position and vice versa.

In an alternate embodiment of the present invention, the tips 15 and 16 can be substituted by other type, for example; pointed needle holders, forceps, scissors type, blade type, plier's type, retractor type, compression type, holder type or any other medical material tip of used nowadays or to be developed.

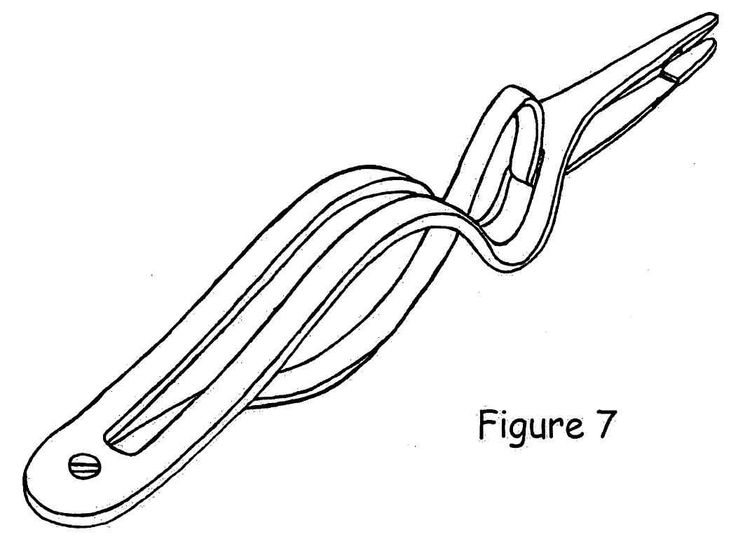

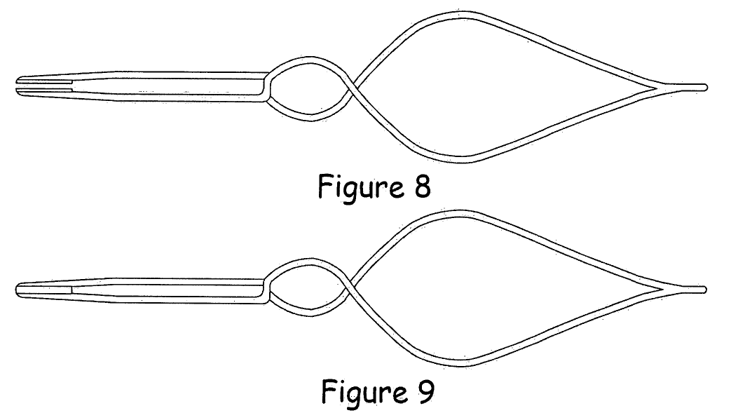

Another alternative embodiment of the present invention, the blocking mechanism can be substituted for any other type of existing mechanism in the State of the Art or any to be developed. The same way the grasping system of the present invention can disregard the blocking system and can be used with any other type of tips such as; pointed needle holders, by forceps, scissors type, blade type, plier's type, retractor type, compression type, holder type. An instrument of this type is shown in FIGS. 7 to 9.

Based on the above revelation, some embodiments and details have been described in order to illustrate the present invention, and will be evident for experts in the subject that variations and modifications can be made without deflecting the range of the present invention. The present technology and new method set apart, for the easy manufacturing of its parts and efficiency when holding during the use of the instrument.

1. A medical instrument with an ambidextrous grasping system, including:
an elongated first arm that has a front portion, an intermediate portion, a grasping area and a rear end;
an elongated second arm that has a front portion, an intermediate portion, a grasping area and a rear end, where both arms are joint between them by joining means in the rear ends;
a sliding blocking mechanism that fits and unfits with a vertical movement performed by the user by pressing on the grasping area;
medical use tips located on the frontal end, in the front portions of both arms;
characterized in that:
the first arm is flat in the front and intermediate portions, from a certain point it curves upward defining a first curved portion in a “U” upside down form, from a second point it curves downward defining a second curved portion in a “U” form, being the second curved portion more extended than the first curved portion, and ends with a flat ending;
the second arm is flat in the frontal and intermediate portions, from a first point it curves downward, defining a first curved portion in a “U” form, from a second point it curves upward defining a second curved portion in a “U” upside down form, being the second curved portion more extended than the first curved portion, and ends with a flat ending;
the second curved portions of both arms, form the grasping area of the instrument;
the first arm separates in two branch lines at the beginning of the first curved portion on a “U” upside down forming a space between the two branch lines, the second arm passes through the space between both branch lines generating a first crossing point;
the second arm passes again through the space formed between both branch lines of the first arm generating a second crossing point;
where in the first crossing point and in the second crossing point both arms do not make any contact or rub;
the blocking mechanism is disposed in the intermediate portion of the instrument located between the frontal portion and the first crossing point,
the blocking mechanism is activated by a slight pressure performed by the user on the grasping area to activate and deactivate the mechanism, keeping a constant distance between both arms in the grasping area to improve the comfort of the user.

2. The medical instrument with an ambidextrous grasping system of claim 1, characterized in that the external faces of both arms in the grasping area possess a knurled grasping to improve the grasping.

3. The medical instrument with an ambidextrous grasping system of claim 1, characterized in that the joining means can preferably be bolts, rivets or set screws, can be used other joining means.

4. The medical instrument with an ambidextrous grasping system of claim 1, characterized in that it comprises a flux material that covers the rear end completely and fastens both arms.

5. The medical instrument with an ambidextrous grasping system of claim 4, characterized in that the flux material can be removed easily for maintenance and graduating the medical instrument.

6. The medical instrument with an ambidextrous grasping system of claim 1, characterized in that the tips can be substituted for another kind of tips such as needle holder, forceps, scissors, knife, pliers type, retractor type, compression type, holder or any other type of medical material tip used nowadays or to be developed.

7. The medical instrument with an ambidextrous grasping system of claim 1, characterized in that the blocking mechanism can be substituted for another type of existing mechanism in the State of the Art or one to be developed.

8. An ambidextrous grasping system, including:
an elongated first arm that has a front portion, an intermediate portion, a grasping area and a rear end;
an elongated second arm that has a front portion, an intermediate portion, a grasping area and a rear end, where both arms are joint between them by joining means in the rear ends;
a sliding blocking mechanism that fits and unfits with a vertical movement performed by the user by pressing on the grasping area;
characterized in that:
the first arm is flat in the front and intermediate portions, from a certain point it curves upward defining a first curved portion in a “U” upside down form, from a second point it curves downward defining a second curved portion in a “U” form, being the second curved portion more extended than the first curved portion, and ends with a flat ending;
the second arm is flat in the frontal and intermediate portions, from a first point it curves downward, defining a first curved portion in a “U” form, from a second point it curves upward defining a second curved portion in a “U” upside down form, being the second curved portion more extended than the first curved portion, and ends with a flat ending;
the second curved portions of both arms, form the grasping area of the instrument;
the first arm separates in two branch lines at the beginning of the first curved portion on a “U” upside down forming a space between the two branch lines, the second arm passes through the space between both branch lines generating a first crossing point;
the second arm passes again through the space formed between both branch lines of the first arm generating a second crossing point;
where in the first crossing point and in the second crossing point both arms do not make any contact or rub;
the blocking mechanism is disposed in the intermediate portion of the instrument located between the frontal portion and the first crossing point,
the blocking mechanism is activated by a slight pressure performed by the user on the grasping area to activate and deactivate the mechanism, keeping a constant distance between both arms in the grasping area to improve the comfort of the user.

9. The ambidextrous grasping system of claim 8, characterized in that the external faces of both arms in the grasping area possess a knurled grasping to improve the grasping.

10. The ambidextrous grasping system of claim 8, characterized in that the joining means can preferably be bolts, rivets or set screws, can be used other joining means.

11. The ambidextrous grasping system of claim 8, characterized in that it comprises a flux material that covers the rear end completely and fastens both arms.

12. The ambidextrous grasping system of claim 11, characterized in that the flux material can be removed easily for maintenance and graduating the medical instrument.

13. The ambidextrous grasping system of claim 8, characterized in that the blocking mechanism can be substituted for another type of existing mechanism in the State of the Art or one to be developed.

14. A medical instrument with an ambidextrous grasping system, including:
an elongated first arm that has a front portion, an intermediate portion, a grasping area and a rear end;
an elongated second arm that has a front portion, an intermediate portion, a grasping area and a rear end, where both arms are joint between them by joining means in the rear ends;
medical use tips located on the frontal end, in the front portions of both arms;
characterized in that:
the first arm is flat in the front and intermediate portions, from a certain point it curves upward defining a first curved portion in a “U” upside down form, from a second point it curves downward defining a second curved portion in a “U” form, being the second curved portion more extended than the first curved portion, and ends with a flat ending;
the second arm is flat in the frontal and intermediate portions, from a first point it curves downward, defining a first curved portion in a “U” form, from a second point it curves upward defining a second curved portion in a “U” upside down form, being the second curved portion more extended than the first curved portion, and ends with a flat ending;
the second curved portions of both arms, form the grasping area of the instrument;
the first arm separates in two branch lines at the beginning of the first curved portion on a “U” upside down forming a space between the two branch lines, the second arm passes through the space between both branch lines generating a first crossing point;
the second arm passes again through the space formed between both branch lines of the first arm generating a second crossing point;
where in the first crossing point and in the second crossing point both arms do not make any contact or rub;
wherein the instrument is opened or closed by a slight pressure performed by the user on the grasping area, keeping a constant distance between both arms in the grasping area to improve the comfort of the user.

15. The medical instrument with an ambidextrous grasping system of claim 14, characterized in that the external faces of both arms in the grasping area possess a knurled grasping to improve the grasping.

16. The medical instrument with an ambidextrous grasping system of claim 14, characterized in that the joining means can preferably be bolts, rivets or set screws, can be used other joining means.

17. The medical instrument with an ambidextrous grasping system of claim 14, characterized in that it comprises a flux material that covers the rear end completely and fastens both arms.

18. The medical instrument with an ambidextrous grasping system of claim 17, characterized in that the flux material can be removed easily for maintenance and graduating the medical instrument.

19. The medical instrument with an ambidextrous grasping system of claim 14, characterized in that the tips can be substituted for another kind of tips such as needle holder, forceps, scissors, knife, pliers type, retractor type, compression type, holder or any other type of medical material tip used nowadays or to be developed.
